(19) United States
(12) Patent Application Publication
(10) Pub. No.: US 2026/0207454 A1
(43) Pub. Date: Jul. 23, 2026

(54) DISCOLORING COMPOSITION FOR KERATIN FIBERS DYED WITH OXIDATIVE DYES OR DIRECT DYES

(71) Applicant: KAO CORPORATION, Tokyo (JP)

(72) Inventors: Axel BALKE, Darmstadt (DE); Marine DELBE, Darmstadt (DE); Jens HEILMANN, Darmstadt (DE)

(73) Assignee: KAO CORPORATION, Tokyo (JP)

(21) Appl. No.: 19/133,503

(22) PCT Filed: Nov. 28, 2023
(86) PCT No.: PCT/EP2023/083299

§ 371 Date: May 28, 2025

(30) Foreign Application Priority Data
Nov. 29, 2022 (EP) 22210171.9

(51) Int. Cl. A61K 8/46; A61Q 5/08

(52) U.S. Cl. CPC A61K 8/46; A61K 8/466; A61Q 5/08; A61K 2800/31

(57) ABSTRACT

A composition for discoloring keratin fibers dyed with oxidative dyes and/or direct dyes, preferably human keratin fibers, more preferably human hair, that includes a) one or more organic reducing agent(s) having at least one thiol group(s), and/or their salt(s), and/or their mixtures, b) one or more organic sulfinic acid(s), and/or their salt(s), and/or their mixtures.

### FIELD OF THE INVENTION

The present invention is directed to a cosmetic composition for discoloring keratin fibers having been dyed with oxidative dyes or directed dyes. A ready-to-use composition, kit-of-parts and bleaching method is disclosed.

### BACKGROUND OF THE INVENTION

Consumers demand long-lasting high-performance products for perfectly covering gray hair. Such products typically comprise oxidative hair dyes. However, fashion trends are quickly changing, and the long lastingness of oxidative hair dyes may be perceived as a disadvantage for customers desiring a new look instantly. Therefore, there is a need for products being able to remove oxidative and direct hair dyes.

Conventional color removing products use the so-called “bleach wash” method, i.e., the oxidative destruction of oxidative hair dyes with the help of persulfates and peroxides. Unfortunately, these techniques confer a high degree of damage to the hair fibers. Moreover, these techniques always lead to significant lightening of the hair because the natural melanin is destroyed along with the artificial dyes.

Contrary to the oxidative color removal, reductive color removal techniques use reducing agents which are more selective to artificial dyes. The multimeric oxidative dyes within the hair fibers are reduced to monomers and can consequently be washed out of the fibers due to the smaller molecular weight of monomers. The oxidative damage is low and there is no destruction of the natural melanin. Thus, no additional lightening of the hair appears.

Unfortunately, the performance of the reductive techniques is lower in comparison to the oxidative ones, and, therefore, there is a real need to develop reductive color removing compositions with improved performance.

### SUMMARY OF THE INVENTION

The first object of the present invention is a composition for discoloring keratin fibers dyed with oxidative dyes and/or direct dyes, preferably human keratin fibers, more preferably human hair, comprising:
a) one or more organic reducing agent(s) having at least one thiol group(s), and/or their salt(s), and/or their mixtures,
b) one or more organic sulfinic acid(s), and/or their salt(s), and/or their mixtures.

The second object of the present invention is a powder composition for discoloring keratin fibers dyed with oxidative dyes and/or direct dyes, preferably human keratin fibers, more preferably human hair, as defined above comprising:
cysteine and/or its salts at a total concentration in the range of 35% to 50% by weight, calculated to the total weight of the composition,
disodium-2-hydroxy-2-sulfinatoacetate at a total concentration in the range of 10% to 25% by weight, calculated to the total weight of the composition,
optionally disodium-2-hydroxy-2-sulfonatoacetate at a total concentration in the range of 5% to 20% by weight, calculated to the total weight of the composition,
optionally one or more pulverulent excipient.

The third object of the present invention is an aqueous ready-to-use discoloring composition for keratin fibers dyed with oxidative dyes and/or direct dyes, preferably human keratin fibers, more preferably human hair, having a pH in the range of 1 to 9, preferably in the range of 1 to 7, more preferably in the range of 1.5 to 5, and comprising the composition as defined above and water at a total concentration of 50% by weight or more, calculated to the total weight of the ready-to-use composition.

The fourth object of the present invention is a kit-of-parts for discoloring keratin fibers dyed with oxidative dyes and/or direct dyes, preferably human keratin fibers, more preferably human hair, comprising:
a composition A comprising one or more compound(s) according to group a) as defined above,
a composition B comprising one or more compound(s) according to groups b) and c) as defined above.

The fifth object of the present invention is a method for discoloring keratin fibers dyed with oxidative dyes and/or direct dyes, preferably human keratin fibers, more preferably human hair, comprising the following steps:
i) mixing and/or dispersing and/or dissolving the composition as defined above or mixing compositions A and B as defined above with an aqueous composition, preferably with water, more preferably with tap water, to yield an aqueous ready-to-use discoloring composition having a pH in the range of 1 to 9,
ii) applying the ready-to-use discoloring composition onto keratin fibers and leaving it for a time period in the range of 1 to 60 min,
iii) rinsing-off the ready-to-use discoloring composition with water and optionally drying the keratin fibers.

### DETAILED DESCRIPTION OF THE INVENTION

The inventors of the present invention have surprisingly and unexpectedly found that compositions, kits, and methods according to the claims of the present invention yielded a synergistic, superior discoloration effect on keratin fibers being dyed with oxidative and/or direct dyes. In addition, the washout of the dyes from the fibers was enhanced and the damage to the fibers was minimal.

### Composition for Discoloring Dyed Keratin Fibers

The present invention is directed to a composition for discoloring keratin fibers dyed with oxidative dyes and/or direct dyes, preferably human keratin fibers, more preferably human hair, comprising:
a) one or more organic reducing agent(s) having at least one thiol group(s), and/or their salt(s), and/or their mixtures,
b) one or more organic sulfinic acid(s), and/or their salt(s), and/or their mixtures.

The composition of the present invention comprises one or more organic reducing agent(s) having at least one thiol group(s), and/or their salt(s), and/or their mixtures, as compound(s) according to group a).

The term ‘thiol group’ has its common meaning in the field of chemistry, i.e., it is an unmodified thiol group able to act as reducing moiety within the molecule of the organic compound.

It is preferred from the viewpoint of cosmetic acceptability that one or more compound(s) according to group a) is cysteine, cystine, cysteamine, thioglycolic acid, thiolactic acid, and/or their salt(s), and/or their mixtures.

The most preferred compound according to group a) is cysteine and/or its salt(s), from the viewpoint of commercial availability.

The composition of the present invention comprises one or more organic sulfinic acid(s), and/or their salt(s), and/or their mixtures, as compound(s) according to group b).

Suitably, one or more compound(s) according to group b) is according to the following general structure:

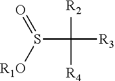

With the provision that R₁ is selected from H, NH₄⁺ or a metal cation, R₂ is selected from OH, NR₅R₆ in which R₅ and R₆, which may be identical or different, are chosen from H or C₁-C₆ alkyl, R₃ is selected from H, alkyl, alkenyl, cycloalkyl or aryl which is/are unsubstituted or substituted by 1 to 3 identical or different substituents chosen from OH, C₁-C₆ alkyl, O—C₁-C₆ alkyl, halogen or CF₃, R₄ is chosen from a radical COOR₁, SO₃R₁, COR₅, CONR₅R₆ or COOR₅ in which R₁, R₅, and R₆ have the denotations as above, or R₄ denotes H in case R₃ denotes an aryl group, in particular an aryl group substituted as described above, and/or their salt(s), and/or their mixtures. Suitable metal cations are all cosmetically acceptable cations such as sodium, potassium, magnesium, and/or calcium.

Suitable examples for compound(s) according to group b) are furyl-(amino)-methanesulfinic acid, amino-(thien-2-yl)-methanesulfinic acid, amino-(1H-imidazol-2-yl)-methanesulfinic acid, amino-(1,3-thiazol-2-yl)-methanesulfinic acid, amino-(1,3-oxazol-2-yl)-methanesulfinic acid, amino-(1H-pyrrol-2-yl)-methanesulfinic acid, amino-(hydroxyl-2-yl)-methanesulfinic acid, amino-(hydroxyl-4-yl)-methanesulfinic acid, amino-[3-hydroxy-5-(hydroxymethyl)-2-methylpyridin-4-yl]-methanesulfinic acid, amino-(quinolin-2-yl)-methanesulfinic acid, amino-(quinolin-4-yl)-methanesulfinic acid, 1H-Benzimidazol-2-yl-(amino)-methanesulfinic acid, 1,3-benzothiazol-2-yl-(amino)-methanesulfinic acid, 1,3-benzoxazol-2-yl-(amino)-methanesulfinic acid, hydroxy-(thien-2-yl)-methanesulfinic acid, hydroxy-(thien-3-yl)-methanesulfinic acid, 3-bromothien-2-yl)-(hydroxyl)-methanesulfinic acid, hydroxy-(1H-imidazol-2-yl)-methanesulfinic acid, hydroxy-(1H-imidazol-5-yl)-methanesulfinic acid, hydroxy-(1-methyl-5-nitro-1H-imidazol-2-yl)-methanesulfinic acid, hydroxy-(1,3-thiazol-2-yl)-methanesulfinic acid, hydroxy-(1,3-oxazol-2-yl)-methanesulfinic acid, hydroxy-(1H-pyrrol-2-yl)-methanesulfinic acid, hydroxy-(hydroxyl-2-yl)-methanesulfinic acid, hydroxy-(hydroxyl-4-yl)-methanesulfinic acid, hydroxy-[3-hydroxy-5-(hydroxymethyl)-2-methylpyridin-4-yl]-methanesulfinic acid, hydroxy-(quinolin-2-yl)-methanesulfinic acid, hydroxy-(quinolin-4-yl)-methanesulfinic acid, 1H-benzimidazol-2-yl-(hydroxyl)-methanesulfinic acid, 1,3-benzothiazol-2-yl-(hydroxyl)-methanesulfinic acid, 1,3-Benzoxazol-2-yl-(hydroxy)-methanesulfinic acid, 1,2-dihydroxyethane-1,2-disulfinic acid, 1,3-dihydroxypropane-1,3-disulfinic acid, hydroxy-{4-[hydroxy(sulfino)methyl]-phenyl}-methanesulfinic acid, {2-chloro-3-[hydroxy-(sulfino)-methyl]-cyclohexyl}-(hydroxy)-methanesulfinic acid, {2-chloro-3-[hydroxyl(sulfino)methyl]cyclopentyl}-(hydroxyl)-methanesulfinic acid, Hydroxy-{5-[hydroxy-(sulfino)-methyl]thien-2-yl}-methanesulfinic acid, hydroxy-{2-[hydroxy(sulfino)-methyl]phenyl}-methanesulfinic acid, hydroxy-{3-[hydroxy-(sulfino)-methyl]-phenyl}-methanesulfinic acid, 1,5-dihydroxypentane-1,5-disulfinic acid, 4-hydroxy-4-sulfinobutanoic acid, 1-Hydroxy-4-methoxy-4-oxobutane-1-sulfinic acid, 1-Hydroxy-4-ethoxy-4-oxobutane-1-sulfinic acid, 4-Hydroxy-4-sulfinopentanoic acid, 2-Hydroxy-5-methoxy-5-oxopentane-2-sulfinic acid, 2-Hydroxy-5-ethoxy-5-oxopentane-2-sulfinic acid, 4-Hydroxy-4-sulfinobut-2-enoic acid, 1-Hydroxy-4-methoxy-4-oxobut-2-ene-1-sulfinic acid, 1-Hydroxy-4-ethoxy-4-oxobut-2-ene-1-sulfinic acid, 4-hydroxy-4-sulfinopent-2-enoic acid, 2-Hydroxy-5-methoxy-5-oxopent-3-ene-2-sulfinic acid, 2-Hydroxy-5-ethoxy-5-oxopent-3-ene-2-sulfinic acid, 5-Hydroxy-5-sulfinopentanoic acid, 1-Hydroxy-5-methoxy-5-oxopentane-1-sulfinic acid, 1-Hydroxy-5-ethoxy-5-oxopentane-1-sulfinic acid, 5-hydroxy-5-sulfinohexanoic acid, 2-Hydroxy-6-methoxy-6-oxohexane-2-sulfinic acid, 2-Hydroxy-6-ethoxy-6-oxohexane-2-sulfinic acid, 4-[Hydroxy-(sulfino)-methyl]-benzoic acid, 4-[amino-(sulfino)-methyl]-benzoic acid, {4-[Hydroxy-(sulfino)-methyl]-phenoxy}-acetic acid, {4-[amino-(sulfino)-methyl]-phenoxy}-acetic acid, 3-Hydroxy-4-[hydroxy-(sulfino)-methyl]-benzoic acid, 3-Hydroxy-4-[amino-(sulfino)-methyl]-benzoic acid, (4-cyanophenyl)-(hydroxy)-methanesulfinic acid, [(2-Methoxy-2-oxoethyl)-amino]-(oxo)-methane-sulfinic acid, 2-Hydroxy-2-sulfinoacetic acid, 2-amino-2-sulfino-acetic acid, (4-cyanophenyl)-(amino)-methane-sulfinic acid,(4-nitrophenyl)-(hydroxy)-methane-sulfinic acid, (4-nitrophenyl)-(amino)-methane-sulfinic acid, 4-hydroxy-1,1-dimethyl-4-sulfinopiperidinium, 1-allyl-4-hydroxy-1-methyl-4-sulfinopiperidinium, 2-Hydroxy-2-sulfino-propionic acid, 2-amino-2-sulfino-propionic acid, 2-Hydroxy-2-sulfinato-acetic acid ethyl ester, 2-Amino-2-sulfinato-acetic acid ethyl ester, 4-Hydroxy-1,1-dimethylpiperidinium-4-sulfinate,1-Allyl 4-hydroxy-1-methylpiperidinium-4-sulfinate, 1-Hydroxy-2-(trimethylammonio)-ethane-sulfinate, Hydroxy-[4-(trimethylammonio)-phenyl]-methane-sulfinate, 4-[Hydroxy-(sulfino)-methyl]-benzene-sulfonic acid, 2-[Hydroxy-(sulfino)-methyl]-benzene-sulfonic acid, 3-Hydroxy-3-sulfinato-propionic acid, 3-amino-3-sulfinato-propionic acid, 1-Hydroxy-2-methoxy-2-oxoethanesulfinic acid, 2-amino-1-hydroxy-2-oxoethanesulfinic acid, 2-(dimethylamino)-1-hydroxy-2-oxoethanesulfinic acid, hydroxy-(sulfino)-methane-sulfonic acid, Hydroxy-(phenyl)-methane-sulfinic acid, hydroxy-(4-hydroxyphenyl)-methanesulfinic acid, hydroxy-(4-methoxyphenyl)-methane-sulfinic acid, and/or their salt(s), and/or their mixtures.

Preferably, form the viewpoint of reducing power, one or more compound(s) according to group b) has the following structure:

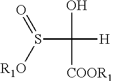

With R₁ having the same denotation as above.

The most preferred compound(s) according to group b) is/are 2-hydroxy-2-sulfinoacetic acid, and/or its salts, preferably it is disodium-2-hydroxy-2-sulfinatoacetate.

It is further preferred that the molar ratio of compound(s) according to group a) to compound(s) according to group b) is in the range of 10:0.1 to 0.1:10, preferably in the range of 3:1 to 1:4, more preferably it is in the range of 1.5:1 to 1:1.5. It is preferred from the viewpoint of stability and acidity that the composition of the present invention comprises one or more compound(s) as compound(s) according to group c) having the following general structure:

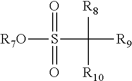

With the provision that R₇ is selected from H, NH₄⁺ or a metal cation, Rₐ is selected from OH, NR₁₁R₁₂ in which R₁₁ and R₁₂, which may be identical or different, are chosen from H or C₁-C₆ alkyl, R₉ is selected from H, alkyl, alkenyl, cycloalkyl or aryl which is/are unsubstituted or substituted by 1 to 3 identical or different substituents chosen from OH, C₁-C₆ alkyl, O—C₁-C₆ alkyl, halogen or CF₃, R₁₀ is chosen from a radical COOR₇, SO₃R₇, COR₁₁, CONR₁₁R₁₂ or COOR₁₁ in which R₇, R₁₁ and R₁₂ have the denotations as above, or R₁₀ denotes H when R₉ denotes an aryl group, in particular an aryl group substituted as described above, and/or their salt(s), and/or their mixtures, preferably one or more compound(s) according to group b) has the following structure:

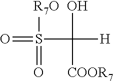

With R₇ having the same denotation as above, more preferably one or more compound(s) according to group c) is disodium-2-hydroxy-2-sulfonatoacetate.

Composition with 10% by Weight of Water or Less

In one aspect of the present invention, it is preferred from the viewpoint of stability and transportation costs that the composition comprises water at 10% by weight or less, preferably at 1% by weight or less, more preferably at 0.1% by weight or less, calculated to the total weight of the composition, more preferably it is anhydrous.

For this aspect, it is preferred from the viewpoint of discoloring power that the total concentration of one or more compound(s) according to group a) is 1% by weight or more, preferably 2% by weight or more, more preferably 3% by weight or more, still more preferably 5% by weight or more, calculated to the total weight of the composition.

It is preferred from the viewpoint of discoloring power and cosmetic acceptability that the total concentration of one or more compound(s) according to group a) is 60% by weight or less, preferably 50% by weight or less, more preferably 45% by weight or less, calculated to the total weight of the composition.

For attaining the above-mentioned effects, it is preferred that the total concentration of one or more compound(s) according to group a) is in the range of 1% to 60% by weight, preferably in the range of 2% to 50% by weight, more preferably in the range of 3% to 45% by weight, still more preferably in the range of 5% to 45% by weight, calculated to the total weight of the composition.

It is preferred from the viewpoint of reducing power that the total concentration of compound(s) according to b) is 0.5% by weight or more, preferably 0.75% by weight or more, more preferably 1% by weight or more, still more preferably 5% by weight or more, calculated to the total weight of the composition.

It is preferred from the viewpoint of cosmetic acceptance that the total concentration of compound(s) according to b) is 35% by weight or less, preferably 30% by weight or less, more preferably 25% by weight or less, calculated to the total weight of the composition.

For attaining the above-mentioned effects, it is preferred that the total concentration of one or more compound(s) according to group b) is in the range of 0.5% to 35% by weight, preferably in the range of 0.75% to 30% by weight, more preferably in the range of 1% to 25% by weight, still more preferably in the range of 5% to 25% by weight, calculated to the total weight of the composition.

It is further preferred from the viewpoint of reducing power that the total concentration of compound(s) according to a) and b) in the composition of the present invention is 40% by weight or more, preferably 50% by weight or more, more preferably 55% by weight or more, calculated to the total weight of the composition.

It is preferred from the viewpoint of stability that the total concentration of one or more compound(s) according to group c) is 1% by weight or more, preferably 2% by weight or more, more preferably 5% by weight or more, still more preferably 10% by weight or more, calculated to the total weight of the composition.

It is preferred from the viewpoint of acidity that the total concentration of one or more compound(s) according to group c) is 30% by weight or less, preferably 25% by weight or less, more preferably 20% by weight or less, calculated to the total weight of the composition.

The composition according to any of the preceding claims characterized in that the total concentration of one or more compound(s) according to group c) is in the range of 1% to 30% by weight, preferably in the range of 2% to 25% by weight, more preferably in the range of 5% to 20% by weight, still more preferably in the range of 10% to 20% by weight, calculated to the total weight of the composition.

Preferably the composition of the present invention is a powder composition.

A powder composition within the meaning of the present invention is any solid composition at 25° C. and atmospheric pressure that consists of freely flowing individual particles/aggregates. A powder composition may comprise liquid agents, though. For example, to make the powder dust-free, it may comprise lipophilic compounds being liquid at 25° C. and atmospheric pressure at a low percentage. The presence of the lipophilic compounds does not alter the overall state of a powder.

Hence, the composition of the present invention may optionally comprise one or more pulverulent excipient as compound(s) according to group d), preferably one or more compound(s) according to group d) is cellulose, chemically modified cellulose fibers, lignin fibers, pectin fibers, natural starch(es), chemically modified starch(es), hydrolyzed natural starch(es), diatomaceous earth, kaolin, bentonite, nylon powder, montmorillonit, gypsum, sawdust and perlite, and/or their mixtures, more preferably one or more compound(s) according to group d) is a natural starch.

Suitable natural starches are potato starch, rice starch, corn starch, tapioca starch, wheat starch, and barley starch, and/or their mixtures.

In case the composition of the present invention comprises one or more compound(s) according to group d), it is preferred that the total concentration of one or more compound(s) according to group d) is 10% by weight, preferably 20% by weight, more preferably 30% by weight, calculated to the total weight of the composition.

In one aspect of the present invention, the composition is a powder composition for discoloring keratin fibers, preferably human keratin fibers, more preferably human hair, dyed with oxidative dyes and/or direct dyes according as defined above comprising:
cysteine and/or its salts at a total concentration in the range of 35% to 50% by weight, calculated to the total weight of the composition,
disodium-2-hydroxy-2-sulfinatoacetate at a total concentration in the range of 10% to 25% by weight, calculated to the total weight of the composition,
optionally disodium-2-hydroxy-2-sulfonatoacetate at a total concentration in the range of 5% to 20% by weight, calculated to the total weight of the composition, and
optionally one or more pulverulent excipient.

### Aqueous Composition

In one aspect of the present invention, the composition is an aqueous composition comprising water at 50% by weight or more, preferably at 60% by weight or more, still more preferably at 70% by weight or more, calculated to the total weight of the composition.

It is preferred from the viewpoint of cosmetic safety that the pH of the composition is 1 or more, preferably 2 or more, still more preferably 2.5 or more.

It is preferred from the viewpoint of stability that the pH of the composition is 6 or less, preferably 5 or less, still more preferably 4.5 or less.

For attaining the above-mentioned effects, it is preferred that the pH of the composition is in the range of 1 to 6, more preferably in the range of 2 to 5, still more preferably in the range of 2.5 to 4.5.

For the aqueous composition it is preferred from the viewpoint of reducing power that the total concentration of one or more compound(s) according to group a) is 0.1% by weight or more, preferably 0.25% by weight or more, more preferably 0.5% by weight or more, still more preferably 1% by weight or more, calculated to the total weight of the composition.

For the aqueous composition it is preferred from the viewpoint of cosmetic safety that the total concentration of one or more compound(s) according to group a) is 20% by weight or less, preferably 15% by weight or less, more preferably 10% by weight or less, still more preferably 7% by weight or less, calculated to the total weight of the composition.

For attaining the above-mentioned effects, it is preferred that the total concentration of one or more compound(s) according to group a) is in the range of 0.1% to 20% by weight, preferably in the range of 0.25% to 15% by weight, more preferably in the range of 0.5% to 10% by weight, still more preferably in the range of 1.0% to 7% by weight, calculated to the total weight of the composition.

For the aqueous composition it is preferred from the viewpoint of reducing power that the total concentration of one or more compound(s) according to group b) is 0.1% by weight or more, preferably 0.25% by weight or more, more preferably 0.5% by weight or more, still more preferably 1% by weight or more, calculated to the total weight of the composition.

For the aqueous composition it is preferred from the viewpoint of cosmetic safety that the total concentration of one or more compound(s) according to group b) is 20% by weight or less, preferably 15% by weight or less, more preferably 10% by weight or less, still more preferably 7% by weight or less, calculated to the total weight of the composition.

For attaining the above-mentioned effects, it is preferred that the total concentration of one or more compound(s) according to group b) is in the range of 0.1% to 20% by weight, preferably in the range of 0.25% to 15% by weight, more preferably in the range of 0.5% to 10% by weight, still more preferably in the range of 1.0% to 7% by weight, calculated to the total weight of the composition.

### Additional Reducing Agents

It is further preferred from the viewpoint of discoloring performance that the composition of the present invention comprises one or more additional reducing agent(s) different from the group(s) according to a) and b), preferably the one or more additional reducing agent(s) is ascorbic acid, and more preferably the total concentration of ascorbic acid is in the range of 0.1% to 10% by weight, calculated to the total weight of the composition.

### Thickening Agents

It is preferred from the viewpoint of viscosity that the composition of the present invention comprises a thickening agent, preferably a thickening polymer, more preferably an anionic thickening polymer, still more preferably a carbohydrate-based anionic thickening polymer.

Suitably, the anionic polymer(s) may be selected from xanthan gum, dehydroxanthan gum, hydroxypropylxanthan gum, carboxymethyl cellulose and synthetically modified starch-based polymers such as hydroxypropyl starch phosphate, and/or their mixtures. Equally suitable are alginic acids, sodium alginates, ammonium alginates, calcium alginates, gum arabic, and guar gum, and/or their mixtures.

The preferred thickening polymer for the composition of the present invention are xanthan gum and/or dehydroxanthan gum, from the viewpoint of their biodegradability and low environmental impact.

Suitably, the total concentration of thickening agents in the composition of the present invention is in the range of 0.1% to 30% by weight, preferably in the range of 0.25% to 25% by weight, more preferably in the range of 0.5% to 25% by weight, calculated to the total weight of the composition.

### Surfactants

The composition of the present invention may further comprise one or more surfactant(s), preferably selected from non-ionic surfactants, anionic surfactants, cationic surfactants, and/or amphoteric/zwitterionic surfactants, and/or their mixtures, more preferably selected from anionic surfactants, from the viewpoint of stabilizing the composition and improving wettability and mixability.

Preferably, the anionic surfactants may be selected from ethoxylated or non-ethoxylated alkyl ether sulfate surfactants, alkyl sulfates, ethoxylated and/or non-ethoxylated alkyl carboxylates, ethoxylated or non-ethoxylated amino acid surfactants, and/or their mixtures, and/or their salts.

Suitable examples are alkyl sulfate or preferably ethoxylated alkyl ether sulfate surfactants or mixtures thereof, and/or salts thereof, having an alkyl chain length of C₁₀ to C₂₂ and an ethoxylation degree from 1 to 50.

Suitable non-ionic surfactants may be selected from alkyl polyglycosides, ethoxylated triglycerides, ethoxylated fatty alcohols, ethoxylated fatty acid esters, and/or their mixtures.

Suitable cationic surfactants are quaternary ammonium surfactants having a carbon chain length in the range of C₁₂ to C₂₂ or surfactants having a tertiary amine group and at least one alkyl chain having a carbon chain length in the range of C₁₂ to C₂₂ such as alkylamidoalkylamine surfactants, and/or their salts. Suitable examples are cetrimonium chloride and behentrimonium chloride.

Suitable amphoteric/zwitterionic surfactants are of betaine type. Suitable compounds may be selected from alkyl betaines and/or alkylamido betaines. A preferred compound selected from alkyl betaines is lauryl betaine. A preferred compound selected from alkylamido betaines is cocamidopropyl betaine. The disclosure also relates to the salts of the compounds.

Suitable concentration ranges for surfactants are in the range of 0.1% to 20% by weight, calculated to the total weight of the composition, from the viewpoint of enhancing wettability of keratin fibers, physical stability, and mixability with other compositions.

### Ready-to-Use Composition

The present invention is also directed to an aqueous ready-to-use discoloring composition for keratin fibers, preferably human keratin fibers, more preferably human hair, dyed with oxidative dyes and/or direct dyes having a pH in the range of 1 to 9, preferably in the range of 1 to 7, more preferably in the range of 1.5 to 5, and comprising the composition as defined above and water at a total concentration of 50% by weight or more, calculated to the total weight of the ready-to-use composition.

Suitably, the composition of the anhydrous and/or powder composition of the present invention is dissolved/mixed/dispersed in/with water at a weight ratio in the range of 0.1:1 to 1:1 (composition:water) directly prior to application onto keratin fibers. Thus, the total concentration of water in the ready-to-use composition is 50% by weight or more, preferably 70% by weight or more, more preferably 80% by weight or more, calculated to the total weight of the ready-to-use composition.

Another preferred mixing weight ratio directly prior to application onto keratin fibers of the composition of the present invention with water is 1:1, thereby leading to a total water concentration of 50% by weight, calculated to the total weight of the ready-to-use composition.

The most preferred mixing weight ratio directly prior to application onto keratin fibers of the composition of the present invention with water is 0.2:1, thereby leading to a water concentration of 80% by weight, calculated to the total weight of the ready-to-use composition.

### Kit-of-Parts

The present invention is also directed to a kit-of-parts for discoloring keratin fibers, preferably human keratin fibers, more preferably human hair, dyed with oxidative dyes and/or direct dyes comprising:
a composition A comprising one or more compound(s) according to group a) as defined above,
a composition B comprising one or more compound(s) according to groups b) and c) as defined above.

It is preferred from the viewpoint of stability, that the total concentration of water in each of the compositions A and B is 10% by weight or less, preferably 1% by weight or less, more preferably 0.1% by weight or less, calculated to the total weight of each of the compositions A and/or B, more preferably compositions A and/or B are anhydrous compositions.

Preferably, compositions A and/or B are powder compositions.

It is preferred from the viewpoint of performance of the kit that compositions A and B are mixed/dissolved/dispersed in water directly prior to the application onto keratin fibers. Preferably, the compositions A, B, and water form the ready-to-use composition as defined above.

It is preferred from the viewpoint of cosmetic applicability that compositions A and/or B comprise any of the compounds defined above such as one or more compound(s) according to the groups c) and/or d), or thickening agents. The concentration of the ingredients are the same as specified for the composition of the present invention.

### Method for Discoloring

The present invention is also directed to a method for discoloring keratin fibers, preferably human keratin fibers, more preferably human hair, dyed with oxidative dyes and/or direct dyes comprising the following steps:
i) mixing and/or dispersing and/or dissolving the composition as defined above or mixing compositions A and B as defined above with an aqueous composition, preferably with water, more preferably with tap water, to yield an aqueous ready-to-use discoloring composition having a pH in the range of 1 to 9,
ii) applying the ready-to-use discoloring composition onto keratin fibers and leaving it for a time period in the range of 1 to 60 min,
iii) rinsing-off the ready-to-use discoloring composition with water and optionally drying the keratin fibers.

It is preferred from the viewpoint of reducing power that the pH of the ready-to-use discoloring composition of step i) is in the range of 1 to 7, more preferably in the range of 1.5 to 5.

It is further preferred that the weight ratio of the composition and water in step i) is in the range of 0.1:1 to 1:0.1 (composition:water), more preferably in the range of 0.1:1 to 0.75:1, still more preferably 0.2:1 to 0.5:1, still more preferably it is 0.2:1.

The preferred application time if step ii) is in the range of 2 min to 45 min, more preferably it is in the range of 5 min to 35 min, still more preferably it is in the range of 10 min to 30 min.

The following examples are to illustrate the present invention and not to limit it.

### Examples

The following composition were prepared.

TABLE 1

| | | | | Comparative. | | Comparative | |
|---|---|---|---|---|---|---|---|
| | | Inv. comp. 1 | | comp. 1 | | comp. 2 | |
| | | % by | | % by | | % by | |
| Ingredients | | weight | mM | weight | mM | weight | mM |
| a) | Cysteine HCl•1 H₂0 | 1.66 | 9.5 | 3.33 | 19.0 | — | — |
| b) | Disodium-2-hydroxy-2- | 1.75 | 9.5 | — | — | 3.50 | 19.0 |
| | sulfinatoacetate | | | | | | |

| | | | | |
|---|---|---|---|---|
| — | Molar ratio a):b) | 1 | — | — |
| | Xanthan gum | 2.50 | 2.50 | 2.50 |

| | | |
|---|---|---|
| — | HCl/NaOH | q.s. ad pH 3.2 |
| | Water | Ad 100.0 |

The following molar masses were used for the calculations:
Cysteine HCl·1H₂O, M=175 g/mol
Disodium-2-hydroxy-2-sulfinatoacetate, M=184 g/mol

The following results on dyed keratin fibers were obtained:

TABLE 2

| | | | |
|---|---|---|---|
| Dyeing | | Comparative. | Comparative |
| composition | Inv. comp. 1 | comp. 1 | comp. 2 |

| | |
|---|---|
| # | [% discoloration] |

| | | | |
|---|---|---|---|
| 1 | 29.28 | 11.77 | 18.12 |
| 2 | 39.55 | 15.22 | 32.20 |
| 3 | 62.63 | 21.37 | 51.14 |

The inventive composition yielded a higher degree of discoloration upon application onto dyed keratin fibers at the same total reducing agent concentration of comparative examples 1 and 2.

TABLE 3

| | | Inv. comp. 2 | | Inv. comp. 3 | | Inv. comp. 4 | |
|---|---|---|---|---|---|---|---|
| | | % by | | % by | | % by | |
| Ingredients | | weight | mM | weight | mM | weight | mM |
| a) | Cysteine HCl•1 | 0.835 | 4.8 | 1.11 | 6.4 | 2.19 | 12.6 |
| | H₂0 | | | | | | |
| b) | Disodium-2- | 2.627 | 14.2 | 2.335 | 12.6 | 1.18 | 6.4 |
| | hydroxy-2- | | | | | | |
| | sulfinatoacetate | | | | | | |

| | | | | |
|---|---|---|---|---|
| — | Molar ratio a):b) | 1:4 | 1:3 | 3:1 |
| | Xanthan gum | 2.50 | 2.50 | 2.50 |

| | | |
|---|---|---|
| — | HCl/NaOH | q.s. ad pH 3.2 |
| | Water | Ad 100.0 |

The following results on dyed keratin fibers were obtained:

TABLE 4

| Dyeing | Inv. comp. 2 | Inv. comp. 3 | Inv. comp. 4 |
|---|---|---|---|
| composition # | [% discoloration] | | |
| 1 | 31.06 | 28.63 | 19.88 |
| 2 | 36.67 | 35.51 | 34.96 |

TABLE 5
The following powder composition was prepared.

| | | Inv. comp. 5 |
|---|---|---|
| Ingredients | | [% by weight] |
| a) | Cysteine HCl | 40.0 |
| b) | Disodium-2-hydroxy-2- | 18.0 |
| | sulfinatoacetate | |
| c) | Disodium-2-hydroxy-2- | 12.0 |
| | sulfonatoacetate | |
| — | Citric acid | 17.5 |
| — | Dehydroxanthan gum | 12.5 |

TABLE 6
The following results on dyed keratin fibers were obtained:

| Dyeing | Inv. comp. 5 |
|---|---|
| composition # | [% discoloration] |
| 1 | 47.45 |
| 2 | 53.70 |
| 3 | 57.86 |

### Methods

L*₁, a*₁, b*₁ values of Yak hair streaks (21 cm long, 2 g per bundle) were measured with a Datacolor 45 spectrometer. The hair streaks were shampooed with a commercially available shampoo under the brand name Goldwell Dualsenses Scalp Specialist Deep Cleansing Shampoo.

The following commercially available dyeing compositions were used:

Goldwell Topchic 6N corresponded to dyeing composition #1. It comprised the following oxidative dyes: Toluene-2,5-diamine sulfate, 4-Chlororesorcinol, Resorcinol, m-Aminophenol, 4-Amino-2-Hydroxytoluene.

Goldwell Topchic 6SB corresponded to dyeing composition #2. It comprised the following oxidative and direct dyes: Toluene-2,5-diamine sulfate, Resorcinol, 1,3-Bis-(2,4-Diaminophenoxy) propane HCl, m-Aminophenol, HC Yellow No. 2.

Goldwell Topchic 7RR corresponded to dyeing composition #3. It comprises the following oxidative and direct dyes: 1-Hydroxyethyl-4,5-Diamino Pyrazole Sulfate, m-Aminophenol, Acid Red 52, HC Blue No. 17, 1-Naphthol.

The dye compositions #1-3 were mixed with an oxidizing composition comprising hydrogen peroxide at 6% by weight in a weight ratio of 1:1. The hair streaks were dyed for 30 min at 40° C., then the streaks were rinsed-off with water, shampooed and blow-dried. L*₂, a*₂, b*₂ values were measured with a Datacolor 45 spectrometer. ΔE*_{ab color} was calculated as follows:

$$Δ {E}_{ab color}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

For bleaching/discoloring the hair streaks, the compositions of tables 1 and 3 were applied onto the dyed hair streaks and left for 30 min at 40° C. The compositions of table 5 were dissolved in water at a weight ratio of 1:5 (1 part powder, 4 parts water) to prepare a ready-to-use composition. 2 g of the compositions (tables 1, 3, or ready-to-use composition made from table 5) were applied onto the hair streaks. The streaks were then rinsed-off with water, shampooed and blow-dried. L*₃, a*₃, b*₃ values were measured. ΔE*_{ab discolored} and % discolored was calculated according to the following formula:

$$Δ {E}_{ab discolored}^{*} = \sqrt{{( {L}_{3}^{*} - {L}_{2}^{*} )}^{2} + {( {a}_{3}^{*} - {a}_{2}^{*} )}^{2} + {( {b}_{3}^{*} - {b}_{2}^{*} )}^{2}}$$

$$\% {discolored}_{measured} = 100 \cdot \frac{Δ {E}_{ab discolored}^{*}}{Δ {E}_{ab color}^{*}}$$

1. A composition for discoloring keratin fibers dyed with oxidative dyes and/or direct dyes, comprising:
a) one or more organic reducing agents having at least one thiol group, or a salt thereof,
b) one or more organic sulfinic acids, or a salt thereof,
wherein the one or more compound(s) according to group b) have the following general structure: 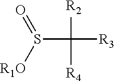
where
R₁ is selected from H, NH₄⁺ and a metal cation,
R₂ is selected from OH, NR₅R₆ in which R₅ and R₆ are each independently chosen from H and C₁-C₆ alkyl,
R₃ is selected from H, alkyl, alkenyl, cycloalkyl and aryl which may be unsubstituted or substituted by 1 to 3 identical or different substituents chosen from OH, C₁-C₆ alkyl, O—C₁-C₆ alkyl, halogen and CF₃,
R₄ is chosen from a radical COOR₁, SO₃R₁, COR₅, CONR₅R₆ and COOR₅ in which R₁, R₅, and R₆ are as described above, or R₄ denotes H when R₃ denotes an aryl group
or a salt thereof,
wherein a molar ratio of a totality of compounds according to group a) to a totality of compounds according to group b) is in the range of 10:0.1 to 0.1:10, and
wherein the one or more compounds according to group a) are selected from the group consisting of cysteine, cystine, cysteamine, thioglycolic acid, thiolactic acid and a salt thereof.

2. The composition according to claim 1, wherein a molar ratio of a totality of compounds according to group a) to a totality of compounds according to group b) is in a range of 3:1 to 1:4.

3. The composition according to claim 1, wherein the one or more compounds according to group a) is cysteine or a salt thereof.

4. The composition according to claim 1, wherein the one or more compounds according to group b) have the following structure: 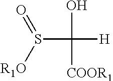
where R₁ is as described above.

5. The composition according to claim 1, wherein the composition comprises water at 10% by weight or less.

6. The composition according to claim 1, wherein a total concentration of the one or more compounds according to group a) is in a range of 1% to 60% by weight, calculated to a total weight of the composition.

7. The composition according to claim 1, wherein a total concentration of one or more compounds according to group b) is in a range of 0.5% to 35% by weight, calculated to a total weight of the composition.

8. The composition according to claim 1, wherein the composition is a powder composition, and optionally comprises one or more pulverulent excipients as compounds according to group d), which is at least one selected from the group consisting of cellulose, chemically modified cellulose fibers, lignin fibers, pectin fibers, natural starch(es), chemically modified starch(es), hydrolyzed natural starch(es), diatomaceous earth, kaolin, bentonite, nylon powder, montmorillonit, gypsum, sawdust and perlite.

9. The composition according to claim 1, wherein the composition is an aqueous composition comprising water at 50% by weight or more, calculated to the total weight of the composition, and having a pH in a range of 1 to 6.

10. The composition according to claim 9, wherein a total concentration of the one or more compounds according to group a) is in a range of 0.1% to 20% by weight, calculated to a total weight of the composition.

11. The composition according to claim 9, wherein a total concentration of the one or more compounds according to group b) is in a range of 0.1% to 20% by weight, calculated to a total weight of the composition.

12. The composition according to claim 1, wherein the composition comprises one or more thickening agents which is a carbohydrate-based anionic thickening polymer.

13. A powder composition for discoloring keratin fibers dyed with oxidative dyes and/or direct dyes according to claim 1, wherein the composition comprises:
cysteine or a salt thereof at a total concentration in a range of 35% to 50% by weight, calculated to a total weight of the composition,
disodium-2-hydroxy-2-sulfinatoacetate at a total concentration in a range of 10% to 25% by weight, calculated to a total weight of the composition,
optionally disodium-2-hydroxy-2-sulfonatoacetate at a total concentration in a range of 5% to 20% by weight, calculated to a total weight of the composition, and
optionally one or more pulverulent excipients.

14. A kit-of-parts for discoloring keratin fibers dyed with oxidative dyes and/or direct dyes, comprising:
a composition A comprising the one or more compounds according to group a) as defined in claim 1, and
a composition B comprising the one or more compounds according to groups b) and c) as defined in claim 1.

15. A method for discoloring keratin fibers dyed with oxidative dyes and/or direct dyes, the method comprising:
i) mixing and/or dispersing and/or dissolving the composition of claim 1 or mixing a composition A comprising the one or more compounds according to group a) as defined in claim 1 and a composition B comprising the one or more compounds according to groups b) and c) as defined in claim 1 with an aqueous composition to yield an aqueous ready-to-use discoloring composition having a pH in the range of 1 to 9,
ii) applying the ready-to-use discoloring composition onto keratin fibers and leaving it for a time period in the range of 1 to 60 min, and
iii) rinsing-off the ready-to-use discoloring composition with water and optionally drying the keratin fibers.

16. The composition according to claim 1, wherein the one or more compounds according to group b) is 2-hydroxy-2-sulfinoacetic acid or a salt thereof.

17. The composition according to claim 1, wherein the composition is anhydrous.

18. The composition according to claim 8, wherein the composition comprises one or more pulverulent excipient as compounds according to group d), selected from the group consisting of cellulose, chemically modified cellulose fibers, lignin fibers, pectin fibers, natural starch(es), chemically modified starch(es), hydrolyzed natural starch(es), diatomaceous earth, kaolin, bentonite, nylon powder, montmorillonit, gypsum, sawdust and perlite.

19. The composition according to claim 1, wherein the compositions has a pH in a range of 1 to 6.

20. The composition according to claim 1, wherein the compositions has a pH in a range of 2.5 to 4.5
